SEQUENCE LISTING (1 sequence)

SEQ ID NO: 1 - protein, 643 aa, Homo sapiens
MEAVETGERPTFGAWDYGVFALMLLVSTGIGLWVGLARGGQRSAEDFFTGGRRLAALPVGLSLSASFMSAVQVLGVPSEAYRYGLKFLWMCLEQLLNSVLTALLFMPVFYRLGLTSTYEYLEMRFSRAVRLCGTLQYIVATMLYTGIVIYAPALILNQVTGLDIWASLLSTGIICTFYTAVGGMKAVVWTDVFQVVVMLSGFWVVLARGVMLVGGPRQVLTLAQNHSRINLMDFNPDPRSRYTFWTFVVGGTLVWLSMYGVNQAQVQRYVACRTEKQAKLALLINQVGLFLIVSSAACCGIVMFVFYTDCDPLLLGRISAPDQYMPLLVLDIFEDLPGVPGLFLACAYSGTLSTASTSINAMAAVTVEDLIKPRLRSLAPRKLVIISKGLSLIYGSACLTVAALSSLLGGGVLQGSFTVMGVISGPLLGAFILGMFLPACNTPGVLAGLGAGLALSLWVALGATLYPPSEQTMRVLPSSAARCVALSVNASGLLDPALLPANDSSRAPSSGMDASRPALADSFYAISYLYYGALGTLTTVLCGALISCLTGPTKRSTLAPGLLWWDLARQTASVAPKEEVAILDDNLVKGPEELPTGNKKPPGFLPTNEDRLFFLGQKELEGAGSWTPCVGHDGGRDQQETNL

(19) United States
(12) Patent Application Publication
(10) Pub. No.: US 2014/0271456 A1
(43) Pub. Date: Sep. 18, 2014

(54) RADIOTRACER IMAGING USING SODIUM IODIDE SYMPORTER POLYPEPTIDES

(71) Applicant: Mayo Foundation for Medical Education and Research, Rochester, MN (US)

(72) Inventors: Stephen James Russell, Rochester, MN (US); Kah-Whye Peng, Rochester, MN (US); Patrycja Lech, Rochester, MN (US)

(73) Assignee: Mayo Foundation for Medical Education and Research, Rochester, MN (US)

(21) Appl. No.: 14/209,369

(22) Filed: Mar. 13, 2014

Related U.S. Application Data
(60) Provisional application No. 61/788,034, filed on Mar. 15, 2013.

(51) Int. Cl. A61K 51/02

(52) U.S. Cl. CPC A61K 51/025
USPC 424/1.11

(57) ABSTRACT

This document provides methods and materials involved in radiotracer imaging using NIS polypeptides. For example, methods and materials for performing imaging techniques that increase the detection sensitivity and resolution of radiotracers localized by NIS reporter gene expression and/or decrease background signals that can be attributed to endogenously expressed NIS polypeptides are provided.

### CROSS-REFERENCE TO RELATED APPLICATIONS

This application claims the benefit of U.S. Provisional Application Ser. No. 61/788,034, filed Mar. 15, 2013. The disclosure of the prior application is considered part of (and is incorporated by reference in) the disclosure of this application.

### 1. TECHNICAL FIELD

This document relates to methods and materials involved in radiotracer imaging using sodium iodide symporter (NIS) polypeptides (e.g., selective radiotracer imaging using NIS polypeptides). For example, this document relates to imaging techniques that increase the detection sensitivity and resolution of radiotracers localized by NIS reporter gene expression and/or decrease background signals that can be attributed to endogenously expressed NIS polypeptides.

### 2. BACKGROUND INFORMATION

NIS polypeptides mediate the uptake and concentration of iodide in the thyroid gland, providing the basis for diagnostic thyroid radioimaging and radioiodine therapy. When nucleic acid encoding a NIS polypeptide is introduced into non-thyroid cells, they can, like thyroid cells, be detected using iodide/pertechnetate radioimaging and/or destroyed using iodide radiotherapy.

SPECT, PET and γ-camera images can be used to monitor the in vivo biodistribution of NIS radiotracers. NIS radiotracers can be efficiently concentrated by genetically modified NIS-transduced cells, but is rapidly removed via efflux from these cells as the blood level of radiotracer falls. The radiotracers can be concentrated in thyroid, stomach, salivary glands, and lactating breast tissue, where NIS is naturally expressed.

### SUMMARY

This document provides methods and materials involved in radiotracer imaging using NIS polypeptides (e.g., selective radiotracer imaging using NIS polypeptides). For example, this document provides methods and materials for performing imaging techniques that increase the detection sensitivity and resolution of radiotracers localized by NIS reporter gene expression and/or decrease background signals that can be attributed to endogenously expressed NIS polypeptides.

As described herein, imaging techniques that involve the uptake of radiotracers such as ¹²³I, ¹²⁴I, ¹²⁵I, B¹⁸F₄ (tetrafluoroborate), and ^{99m}TcO₄ (pertechnetate) by expressed NIS polypeptides can be used for accurate, sensitive, high resolution monitoring of gene expression and cell fate in living mammals. Unlike luciferase and GFP reporter genes, which rely on optical imaging, NIS polypeptides can be used for quantitative expression monitoring in large mammals and humans as well as in deep-seated tissues (e.g., mouse tissues) because, unlike photons, gamma rays are minimally attenuated by the tissues through which they pass. In addition, NIS polypeptides can be used clinically since NIS polypeptide radiotracers are clinically approved, NIS polypeptides lack immunogenicity, and NIS polypeptides are harmless to targeted tissues.

NIS radiotracers secreted into saliva can be swallowed and can move via the esophagus to the stomach. Considerable amounts of iodide can be transported from the bloodstream into the gastric lumen. NIS radiotracers secreted into the stomach can be reabsorbed after passing into the duodenum and jejunum. Most NIS radiotracers are renally excreted. NIS radiotracer images, therefore, can have high background signals in thyroid, stomach, salivary glands, and bladder, with weaker background signals in esophagus, small intestine, kidneys, and blood. This background radioactivity can decrease the sensitivity of the NIS reporter gene imaging system. For example, sensitivity can be severely compromised for regions in close proximity to the stomach, thyroid, and salivary glands. The current limit of detection in a mouse with pinhole collimation on the X-SPECT instrument in regions with low background activity is approximately 2×10⁵ subcutaneously or intratumorally located NIS-expressing cells (clustered).

The methods and materials provided herein can be used to decrease background signals in NIS reporter gene imaging, thereby increasing the sensitivity and resolution of the technology. In some cases, background can be reduced by digital subtraction of perfectly co-registered SPECT images obtained by dual isotope imaging using two chemically distinct NIS radiotracers with different gamma emission spectra. The resolution of such a method can be further enhanced through the use of mutant NIS reporter genes encoding mutant NIS polypeptides with selectively diminished ability to concentrate one or other of the two chemically distinct radiotracers.

In another embodiment, background signals in SPECT and PET imaging can be reduced by co-administering non-radioactive perchlorate anions with a NIS radiotracer, in conjunction with a mutant NIS reporter gene encoding a mutated NIS polypeptide that is relatively resistant to perchlorate inhibition. Methods for isolating a mutated NIS gene encoding a mutated NIS polypeptide having superior performance in the imaging methods are described herein.

In some cases, the methods and materials provided herein can increase detection levels as compared to such a 2×10⁵ cell cluster limit. For example, nucleic acid encoding a mutant NIS polypeptide (e.g., NIS-93E) can be used in place of nucleic acid encoding a wild-type NIS polypeptide. In such cases, two or more radiotracers can be used during an imaging process. For example, one radiotracer can be a radiotracer that is transported into cells endogenously expressing wild-type NIS polypeptides with minimal or no transport into cells expressing the mutant NIS polypeptides. The other radiotracer can be a radiotracer that is transported into cells via both endogenously expressed wild-type NIS polypeptides and expressed mutant NIS polypeptides. In this case, a comparative imaging process can be used such that signals from the radiotracer transported into cells endogenously expressing wild-type NIS polypeptides, with minimal or no transport into cells expressing mutant NIS polypeptides, are removed from those signals obtained from the radiotracer transported into both cells endogenously expressing wild-type NIS polypeptides and cells expressing mutant NIS polypeptides. Such subtraction images can highlight the cells (e.g., infected cells) expressing the mutant NIS polypeptide (e.g., NIS-93E), but not those cells that endogenously express wild-type NIS polypeptides.

In some cases, an oral CT contrast agent (e.g., barium sulfate, gastrografin, or iodinated contrast) can be used to enhance discrimination of NIS polypeptide expression in areas around the stomach (e.g., the liver, perigastric area, pancreas, spleen, kidneys, and pleural space). The stomach endogenously expresses wild-type NIS polypeptides and uptakes radioisotopes such as radioiodine or pertechnetate, making the stomach highly visible in planar γ-camera, SPECT, or PET images. The strong stomach signal can make it difficult to define, detect, or pinpoint other NIS polypeptide-positive cells in the surrounding area (e.g., the liver, perigastric area, pancreas, spleen, kidneys, and pleural space). Oral administration of an oral CT contrast agent (e.g., barium sulfate, gastrografin, or iodinated contrast) followed by imaging can result in reduced detection of radiotracer signals from the stomach, thereby allowing for detection of radiotracer signals from NIS polypeptide-expressing cells in the area around the stomach.

In general, one aspect of this document features a method of imaging an animal comprising cells expressing a NIS transgene. The method comprises, or consists essentially of, (a) administering two or more gamma-emitting NIS radiotracers to the animal, (b) collecting imaging data during a single imaging session using at least two energy windows that distinguish the gamma emissions of the two or more radiotracers to obtain at least two gamma emission datasets, (c) subtracting one of the at least two gamma emission datasets from another of the at least two gamma emission datasets to obtain a resulting dataset capable of being used to generate subtraction images for visual or other analysis. One of the at least two gamma emission datasets can be pseudo-colored in one color, another of the at least two gamma emission datasets is pseudo-colored in another color, and wherein the at least gamma emission datasets are digitally merged to generate one or more pseudo-color images for visual analysis. The NIS transgene can encode a mutated NIS polypeptide with diminished capacity to concentrate one or more NIS radiotracers. The NIS transgene can encode a mutated NIS polypeptide with diminished capacity to concentrate pertechnetate. The mutant NIS polypeptide can be NIS-93E.

In another aspect, this document features a method of imaging an animal comprising cells expressing a NIS transgene. The method comprises, or consists essentially of, (a) administering a gamma-emitting or positron-emitting NIS radiotracer to the animal, (b) administering non-radioactive perchlorate anions to the animal in a dose sufficient to substantially inhibit the uptake of the radiotracer by endogenous NIS-expressing cells, and (c) collecting imaging data from the animal. The NIS transgene can encode a mutated NIS polypeptide whose ability to concentrate NIS radiotracers has reduced susceptibility to perchlorate inhibition in comparison to a non-mutated NIS polypeptide.

In another aspect, this document features an isolated nucleic acid encoding a mutant NIS polypeptide, wherein compared to a non-mutated NIS polypeptide, the mutant NIS polypeptide has substantially reduced capacity to concentrate pertechnetate and maintains at least 30 percent of its capacity to concentrate iodide. The mutant NIS polypeptide can have a greater than 60% reduction in its capacity to concentrate pertechnetate as compared to the non-mutated NIS polypeptide.

In another aspect, this document features an isolated nucleic acid encoding a mutant NIS polypeptide, wherein compared to a non-mutated NIS polypeptide, the ability of the mutant NIS polypeptide to concentrate NIS radiotracers has reduced susceptibility to perchlorate inhibition.

In another aspect, this document features a method for obtaining a mutant NIS polypeptide, wherein the method comprises selecting, from a population of cells expressing different mutant NIS polypeptides and a fluorescent protein biosensor of intracellular iodide concentration, a cell that expresses a mutant NIS polypeptide that comprises the ability to concentrate iodide anions and comprises a reduced ability to concentrate pertechnetate anions. The method can comprise (a) pre-incubating the cells with a stannous pyrophosphate solution, (b) exposing the cells to ^{99m}TcO₄ in a concentration sufficient to kill greater than 99% of cells expressing a non-mutated NIS polypeptide, (c) exposing surviving fluorescent cells to potassium iodide in a concentration sufficient to quench fluorescence, (d) selecting cells whose fluorescence is quenched by the potassium iodide, and (e) obtaining the nucleic acid encoding a mutated NIS polypeptide that is expressed by the selected cells.

In another aspect, this document features a method for obtaining a mutant NIS polypeptide, wherein the method comprises selecting, from a population of cells expressing different mutant NIS polypeptides and a fluorescent protein biosensor of intracellular iodide concentration, a cell that expresses a mutant NIS polypeptide that comprises the ability to concentrate iodide anions in the presence of perchlorate anions in a concentration sufficient to inhibit the uptake of iodide anions by cells expressing a non-mutated NIS polypeptide. The method can comprises (a) simultaneously exposing the cells to perchlorate anions in a concentration sufficient to inhibit the uptake of iodide anions by cells expressing a non-mutated NIS polypeptide and to potassium iodide in a concentration sufficient to quench fluorescence, (b) selecting the cells whose fluorescence is quenched by the potassium iodide, and (c) obtaining the nucleic acid encoding a mutated NIS polypeptide that is expressed by the selected cells.

In another aspect, this document features a vector comprising a nucleic acid provided herein.

In another aspect, this document features a cell comprising the nucleic acid provided herein.

In another aspect, this document features a non-human transgenic animal comprising the nucleic acid provided herein.

In another aspect, this document features a method for imaging a mammal to reduce background from cells endogenously expressing a wild type NIS polypeptide within the stomach of the mammal, wherein the method comprises obtaining an image of radioisotope signals from a radioisotope present within the mammal, wherein the image is obtained within four hours of the mammal ingesting a contrast agent, wherein cells outside the stomach of the mammal expressing a NIS polypeptide uptake the radioisotope. The radioisotope can be pertechnetate or radioiodide. The contrast agent can be barium sulphate.

In another aspect, this document features a method for imaging a mammal to reduce background from radioisotope signals from cells endogenously expressing a wild-type NIS polypeptide, wherein the mammal comprises cells endogenously expressing the wild-type NIS polypeptide and cells expressing a mutant NIS polypeptide. The method comprises (a) obtaining a first image of radioisotope signals from a first radioisotope present within a mammal, wherein cells endogenously expressing the wild-type NIS polypeptide within the mammal uptake the first radioisotope to a greater extent than cells expressing the mutant NIS polypeptide, (b) obtaining a second image of radioisotope signals from a second radioisotope present within a mammal, wherein cells endogenously expressing the wild-type NIS polypeptide within the mammal and cells expressing the mutant NIS polypeptide within the mammal uptake the second radioisotope, and (c) removing radioisotope signals of the first image from the radioisotope signals of the second image to obtain a final image. The mammal can be a human. The wild-type NIS polypeptide can be a human NIS polypeptide. The mutant NIS polypeptide can be a NIS-93E polypeptide or a NIS-93Q polypeptide. The mutant NIS polypeptide can be a NIS-93E polypeptide. The first radioisotope can be pertechnetate. The second radioisotope can be radioiodide. The cells expressing the mutant NIS polypeptide within the mammal can lack the ability to uptake the first radioisotope. The cells endogenously expressing the wild-type NIS polypeptide and cells expressing the mutant NIS polypeptide can uptake the second radioisotope with substantially different efficiencies. The method can comprise removing substantially all radioisotope signals of the first image from the radioisotope signals of the second image to obtain the final image.

In another aspect, this document features a method for imaging a mammal to reduce background from radioisotope signals from cells endogenously expressing a wild-type NIS polypeptide, wherein the mammal comprises cells endogenously expressing the wild-type NIS polypeptide and cells expressing a mutant NIS polypeptide. The method comprises (a) obtaining a first image of radioisotope signals from a first radioisotope present within a mammal, wherein cells endogenously expressing the wild-type NIS polypeptide within the mammal uptake the first radioisotope to a greater extent than cells expressing the mutant NIS polypeptide, (b) obtaining a second image of radioisotope signals from a second radioisotope present within a mammal, wherein cells endogenously expressing the wild-type NIS polypeptide within the mammal and cells expressing the mutant NIS polypeptide within the mammal uptake the second radioisotope, (c) comparing the first image and the second image to identify one or more overlapping radioisotope signals present in the first image and the second image, and (d) removing one or more of the one or more overlapping radioisotope signals from the second image to obtain a final image. The mammal can be a human. The wild-type NIS polypeptide can be a human NIS polypeptide. The mutant NIS polypeptide can be a NIS-93E polypeptide or a NIS-93Q polypeptide. The mutant NIS polypeptide can be a NIS-93E polypeptide. The first radioisotope can be pertechnetate. The second radioisotope can be radioiodide. The cells expressing the mutant NIS polypeptide within the mammal can lack the ability to uptake the first radioisotope. The cells endogenously expressing the wild-type NIS polypeptide and cells expressing the mutant NIS polypeptide can uptake the second radioisotope with substantially different efficiencies. The method can comprise removing substantially all of the overlapping radioisotope signals identified in step (c) from the second image to obtain the final image.

Unless otherwise defined, all technical and scientific terms used herein have the same meaning as commonly understood by one of ordinary skill in the art to which this invention pertains. Although methods and materials similar or equivalent to those described herein can be used to practice the invention, suitable methods and materials are described below. All publications, patent applications, patents, and other references mentioned herein are incorporated by reference in their entirety. In case of conflict, the present specification, including definitions, will control. In addition, the materials, methods, and examples are illustrative only and not intended to be limiting.

The details of one or more embodiments of the invention are set forth in the accompanying drawings and the description below. Other features, objects, and advantages of the invention will be apparent from the description and drawings, and from the claims.

### DESCRIPTION OF DRAWINGS

FIG. 1. NIS mutant G93E selectively transports Iodine-125, but not Technetium-99m. Uptake of Iodine-125 in the absence or presence of perchlorate (competitive inhibitor of uptake) by Mel624 cells stably expressing NIS (A) or NIS-G93E (B) was determined Uptake of Technetiun-99m in the absence or presence of perchlorate via NIS (C) or NIS-G93E (D) was determined

FIG. 2A is a photograph of a strong signal (circled) due to NIS polypeptide-mediated uptake in stomach tissue of a mouse not given an oral contrast agent pre-treatment. It is unclear from the imaging that there are positive signals from surrounding tissues. FIG. 2B is a photograph of the same mouse fed oral contrast agent. The imaging demonstrates the significant reduction of the stomach signal, giving confidence that the positive signals in the surrounding liver tissue are due to NIS polypeptide expression in the NIS transduced liver.

FIG. 3 is a schematic representation of an overall approach and the rationale for selecting NIS mutants that continue to concentrate radioiodine even in the presence of perchlorate concentrations that inhibit iodide transport by wild-type NIS polypeptides.

FIG. 4 is a listing of the amino acid sequence of human NIS-93E (SEQ ID NO:1).

FIG. 5. ¹²⁵I/^{99m}Tc subtraction SPECT. A: Fused ¹²⁵I+^{99m}Tc SPECT/CT emission data. B: Stomach subtraction SPECT dataset. C: Thyroid subtraction SPECT dataset.

FIG. 6. SPECT/CT dual isotope imaging of nude mice implanted with SQ BXPX-3-hNIS tumors. Animals were injected i.p with 250 μCi ¹²⁵I and 250 μCi ^{99m}Tc and imaged four hours later. Images were processed using Vivo Quant software. The scale was adjusted manually to get minimal yellow in the tissues. Signal from ^{99m}Tc (green) (A) and ¹²⁵I (red) (B) were visible in the thyroid, stomach, and tumor. Dual isotope imaging (C) shows that four hours after isotope administration the thyroid preferentially accumulates ¹²⁵I (red), the stomach preferentially accumulates ^{99m}Tc (green), and the tumor does not discriminate between the two isotopes (yellow).

### DETAILED DESCRIPTION

This document provides methods and materials involved in radiotracer imaging using NIS polypeptides. For example, this document provides methods and materials for performing imaging techniques that increase the detection sensitivity and resolution of radiotracers localized by NIS reporter gene expression and/or decrease background signals that can be attributed to endogenously expressed NIS polypeptides.

### Comparison of Serial Images to Address the Background Problem

Background signal reduction/negation can increase the sensitivity of NIS reporter gene imaging. One approach to this problem is to compare images obtained under standard conditions before and after gene transfer/cell implantation as described elsewhere (Iskandrian et al., J. Nucl. Cardiol., 16:6-9 (2009)). NIS-transduced cells can then be identified as areas of increased signal intensity compared to baseline images. Comparing images by visual inspection, however, is relatively insensitive because it may be difficult to discern subtle changes in signal intensity caused by a small number of NIS-transduced cells against the background radiotracer signals from blood pool, renal excretion, or small intestinal residence as well as NIS-dependent thyroid, stomach, salivary, and breast uptake. Since SPECT and PET images are digital, the possibility of digitally subtracting the baseline image from the post-treatment image can be considered. This approach, however, can be subject to a large number of confounding variables including (i) image acquisition parameters between serial images, (ii) image quality, (iii) positioning of the subject, (iv) movement artifacts, and (v) metabolic rate (Ritt et al, Eur J. Nucl. Med. Mol. Imaging, 38(Suppl. 1):569-77 (2011)). Thus, despite their theoretical appeal, the superiority of digital versus visual methods to compare serial radiotracer images is not yet proven.

### Dual Isotope Subtraction SPECT

¹²³I/^{99m}Tc subtraction SPECT can be performed using a dual-detector SPECT camera. In this instance, the SPECT images can be perfectly co-registered and can be subtracted from each other. With a SPECT/CT instrument, the subtraction image also can be perfectly co-registered with the CT image, which provides accurate anatomical information. See, e.g., Neumann et al., J. Nucl. Med., 49:2012 (2008).

Imaging Methods with Reduced Background

In one embodiment, background can be reduced by digital subtraction of perfectly co-registered SPECT images obtained by dual isotope imaging using two chemically distinct NIS radiotracers with different gamma emission spectra. Prior to digital image subtraction, normalization factors can be applied to equalize voxel intensities of the datasets for each of the administered radiotracers in the organ whose background image is to be negated.

In another embodiment, the resolution of a method provided herein can be further enhanced through the use of a mutant NIS reporter gene that encodes a mutated NIS polypeptide having a selectively diminished ability to concentrate one or the other of the two chemically distinct radiotracers.

In another embodiment, background signals in SPECT and PET imaging studies can be reduced by co-administration of nonradioactive perchlorate anions with a NIS radiotracer, in conjunction with a mutant NIS reporter gene encoding a mutated NIS polypeptide that is relatively resistant to perchlorate inhibition. Perchlorate-resistant NIS polypeptides can provide a useful means to target the accumulation of therapeutic NIS-specific anions such as ¹³¹I selectively to tumor cells transduced with a gene encoding a perchlorate-resistant NIS polypeptide.

NIS transports several different anions. Some of these anions are available as radioactive tracers (e.g., ¹²³I, ¹²⁴I, ¹²⁵I, ¹³¹I, B¹⁸F₄, ¹⁸⁶ReO₄, ¹⁸⁸ReO₄, ^{99m}TCO₄, ⁹⁴TcO₄, and ³⁶ClO₄) and can be used as radiotracers for imaging NIS-positive tissues such as the thyroid gland. Convenient tracer anions for animal and human imaging applications are radioiodide and pertechnetate, both of which are available with gamma ray energies that differ sufficiently to allow their independent SPECT when administered together to a single experimental subject. In addition, perchlorate can be safely administered to human subjects in a concentration sufficient to inhibit NIS-mediated uptake of NIS-specific radiotracer anions (Rubello et al., Clin. Nucl. Med., 25:527-531 (2000)).

This document is based, at least in part, on the discoveries that important differences exist in the distributions of chemically distinct NIS radiotracer anions in different cells and tissues in the body that permit the identification of NIS transgene-expressing cells and tissues using a dual isotopic imaging methodology provided herein. This document also demonstrates that the differential accumulation of different NIS-specific anionic radiotracers in tissues naturally expressing NIS versus those transduced with a NIS reporter transgene can be further exaggerated by using a NIS transgene encoding a mutated NIS polypeptide with altered anion specificity.

### Genes Encoding Tracer-Selective or Perchlorate-Resistant NIS Mutant Polypeptides

Certain mutated NIS genes have been shown to encode mutant NIS polypeptides that differ from parental NIS in respect of their Kₘ and Vₘₐₓ values for ReO₄ but not for iodide (International Patent Application Publication No. WO 2011/133216 A2). As described herein, an R93E NIS mutant polypeptide can concentrate iodide with greater efficiency than TcO₄. In some cases, mutant NIS polypeptides such as R93E, which preferentially transport iodide over pertechnetate, can be used in place of wild type NIS for the subtractive imaging approaches provided herein.

As described herein, additional NIS mutant polypeptides with optimal characteristics to minimize the background and maximize the signal that can be obtained from NIS gene transduced cells and tissues can be obtained using the methods provided herein. For example, the methods provided herein can be used to isolate nucleic acid molecules encoding NIS polypeptides that continue to concentrate radioiodine efficiently even in the presence of perchlorate concentrations that inhibit iodide transport by non-mutated NIS. In some cases, NIS mutant polypeptides selectively compromised in their ability to transport certain NIS anions while retaining at least 30% of the iodide concentrating power of the wild type NIS can be obtained. In some cases, NIS mutant polypeptides capable of transporting iodide in the presence of perchlorate concentrations that are inhibitory to the iodide transporting activity of the wild type NIS protein can be obtained.

As described herein, nucleic acid molecules encoding mutant NIS polypeptides can be identified by scanning mutagenesis of the NIS polypeptide and subsequent detailed analysis of individual mutants. In some cases, nucleic acid molecules encoding mutant NIS polypeptides can be obtained by generating an expression library of randomly mutated NIS genes using, for example, a library to infect cells and subjecting the cells to a screening method provided herein and designed to isolate cells expressing mutant NIS polypeptides with desired properties. Nucleic acid molecules encoding mutant NIS polypeptides with desired properties can be recovered by PCR amplification from the selected NIS-transduced cells.

The following are representative examples of selection strategies that can be applied to cell populations transduced with expression libraries encoding mutated NIS polypeptides. In both cases, the expression library (which can be, for example, a lentiviral expression library) can be constructed such that each mutated NIS polypeptide is co-expressed in its transduced cell with the YFP-H148Q/I152L halide-sensitive fluorescent reporter protein whose fluorescence can be readily quenched by intracellular iodide. Importantly, while the fluorescence of purified YFP-H148Q/I152L can be quenched with equal efficiency by iodide and perchlorate anions, the perchlorate anions can be concentrated 10-fold less efficiently in NIS expressing cells compared to iodide anions. Hence, perchlorate can be used to block fluorescence quenching by iodide in living NIS-expressing cells (Cianchetta et al., Toxicology and Applied Pharmacology, 243:372-380 (2010)). Thus, a selection method can be performed in a manner that avoids the use of radioactivity.

As described elsewhere, pre-incubation with stannous pyrophosphate solution can cause intensive intracellular retention of ^{99m}TcO₄ anions in NIS-expressing cells in vitro (Wunderlich et al., Nuklearmedizin, 51:179-185 (2012)). Because of their low energy and short path length, the Auger electrons emitted by ^{99m}TcO₄ are unable to kill cells unless they are concentrated intracellularly. Hence, at high concentrations of ^{99m}TcO₄, the viability of NIS-expressing, stannous pyrophosphate exposed cells can be reduced by three to four orders of magnitude due to intracellular concentration of the Auger-emitting radioisotope. Thus, the selective killing of cells whose expressed NIS polypeptides retain the ability to concentrate pertechnetate, and conversely, the selective survival of cells whose expressed NIS polypeptides are compromised in their ability to concentrate pertechnetate can be used to isolate NIS polypeptides compromised in their ability to concentrate pertechnetate.

### Selective Loss of the Ability to Concentrate Pertechnetate Anions

The following can be performed as a method for selecting, from a diversity of cells expressing mutated NIS polypeptides and a fluorescent protein biosensor of intracellular iodide concentration (e.g., YFP-H148Q/I152L), a cell or cells that express a mutant NIS polypeptide that retains the ability to concentrate iodide anions but which has reduced ability to concentrate pertechnetate anions. The method can include the following steps: (i) Optional preincubation of the cells with a stannous pyrophosphate solution to ensure that they will efficiently retain radioactive Auger-emitting pertechnetate anions, (ii) Exposing the cells to ^{99m}TcO₄ in a concentration and duration sufficient to kill greater than 99% of cells expressing a non-mutated NIS protein, (iii) Exposing the surviving YFP fluorescent cells (optionally selected by using a flow cytometric cell sorting approach) to cold potassium iodide in a concentration sufficient to quench their fluorescence. Fluorescence quenching can be determined using a fluorescence microscope or fluorescence plate reader. In some cases, radioiodine in conjunction with an autoradiographic plate reader or scintillation counting can be used to screen for iodide concentrating activity, (iv) Selecting the cells whose fluorescence is quenched by the cold KI, (v) Recovering the sequence of the gene encoding the mutated NIS protein expressed in the selected cells. This last stage can be accomplished by PCR amplification of the mutated NIS gene from the selected cell.

### Selective Resistance to the Inhibitory Effect of Perchlorate Anions

The following can be performed as a method for selecting, from a diversity of cells expressing mutated NIS polypeptides and a fluorescent protein biosensor of intracellular iodide concentration (e.g., YFP-H148Q/I152L), a cell or cells that express a mutant NIS polypeptide that retains the ability to concentrate iodide anions in the presence of perchlorate anions in a concentration sufficient to inhibit the uptake of iodide anions by cells expressing a non-mutated NIS polypeptide. The method can include the follows steps: (i) Exposing the cells to perchlorate anions in a concentration sufficient to inhibit the uptake of iodide anions by cells expressing a non-mutated NIS polypeptide, and simultaneously exposing the cells to cold potassium iodide in a concentration sufficient to quench their fluorescence. The range of appropriate concentrations for each of the anions can be broad (e.g., the range can be between 3 and 300 μM). In some cases, radioiodine in conjunction with an auto-radiographic plate reader or scintillation counting can be used to screen for iodide concentrating activity, (ii) Selecting the cells whose fluorescence is substantially quenched by the iodide, and (iii) Recovering the sequence of the gene encoding the mutated NIS protein expressed in the selected cells.

### Use of Mutant NIS Polypeptides

As described herein, nucleic acid encoding a mutant NIS polypeptide (e.g., NIS-93E) can be used as a reporter when delivering nucleic acid molecules, viruses, viral vectors, or other therapeutic agents to cells within a mammal. For example, an oncolytic virus configured to express nucleic acid encoding a mutant NIS polypeptide can be administered to a mammal under conditions wherein the oncolytic virus infects cancer cells located within the mammal Following administration of the oncolytic virus, a radiotracer or combination of radiotracers can be given to the mammal, and radiotracer imaging techniques can be used to identify those cells within a mammal that were infected with the administered oncolytic virus. In some cases, two or more radiotracers can be used during the imaging process. For example, one radiotracer can be a radiotracer that is transported into cells endogenously expressing wild-type NIS polypeptides with minimal or no transport into cells expressing the mutant NIS polypeptides. The other radiotracer can be a radiotracer that is transported into cells via both endogenously expressed wild-type NIS polypeptides and expressed mutant NIS polypeptides. In this case, a comparative imaging process can be used such that signals from the radiotracers transported into cells endogenously expressing wild-type NIS polypeptides, with minimal or no transport into cells expressing mutant NIS polypeptides, are removed from those signals obtained from the radiotracers transported into both cells endogenously expressing wild-type NIS polypeptides and cells expressing mutant NIS polypeptides. Such subtraction images can highlight the cells (e.g., oncolytic virus infected cells) expressing the mutant NIS polypeptide (e.g., NIS-93E), but not those cells that endogenously express wild-type NIS polypeptides.

Examples of mutant NIS polypeptides include, without limitation, NIS-93E (FIG. 4). Cells expressing nucleic acid encoding NIS-93E can uptake radioiodide with minimal or no uptake of pertechnetate. Other mutant NIS polypeptides can be obtained as set forth in Example 4.

### Oral CT Contrast Agents

In some cases, an oral CT contrast agent (e.g., barium sulfate, iodinated contrast, or gastrografin) can be used to enhance discrimination of NIS polypeptide expression in areas around the stomach. For example, oral administration of an CT contrast agent (e.g., barium sulfate, iodinated contrast, or gastrografin) followed by imaging can result in reduced detection of radiotracer signals (e.g., radioiodide or pertechnetate) from the cells of the stomach that express wild-type NIS polypeptides, thereby allowing for detection of radiotracer signals from NIS polypeptide-expressing cells in areas around the stomach. Examples of such areas include, without limitation, the liver, perigastric area, pancreas, spleen, kidneys, pleural space, and omentum.

The invention will be further described in the following examples, which do not limit the scope of the invention described in the claims.

### EXAMPLES

### Example 1

### Selective Radiotracer Uptake by a Mutant NIS Polypeptide

Mel624 cells stably expressing a wild-type human NIS polypeptide (Mel-NIS) or one of two mutant NIS polypeptides (Mel-NIS-93E or Mel-NIS-93Q) were plated in 96-well plates at a density that would lead them to confluency on the day of the uptake assay. 1× Hank's balanced salt solution with calcium, magnesium (HBSS) (Cellgro) and 1 mM HEPES was adjusted to pH 7.3 using KOH (HBBS-HEPES) on the day of the assay. Culture medium was removed from the cells, and 150 μL of HBSS-HEPES+/−300 μM KClO₄ was added to the wells. The cells were incubated for 10 minutes at room temperature prior to the addition of radiotracer. Radiotracers, ¹²⁵I and ^{99m}Tc, were diluted in HBSS-HEPES to about 70,000 to 100,000 CPM/25 μL (CPM, counts per minute). 25 μL of radiotracer was added to the cells in HBSS-HEPES+/−KClO₄, and the cells were incubated at 37° C. for 1 hour. The cells were then washed twice with cold HBSS-HEPES buffer and lysed to release the intracellular radiotracer by adding 200 μL 1M NaOH. Lysates were transferred to strip tubes and quantified using a gamma counter.

Substantial uptake of both radioiodide and pertechnetate was observed for cells expressing wild-type human NIS polypeptide (Mel-NIS) (FIG. 1). Cells expressing NIS-93E polypeptides were capable of up-taking radioiodide, but not pertechnetate (FIG. 1). These results demonstrate that NIS-93E selectively concentrates radioiodide, but not pertechnetate.

### Example 2

### In Vivo Subtractive Imaging of NIS93E Expressing Tumors to Remove Background Signals from Endogenous NIS Expression in Tissues

Image subtraction is used to discriminate a tumor that concentrates only one of two NIS isotopes from organs that concentrate both isotopes (e.g., thyroid gland, stomach, and salivary glands). BxPC3 cells (human pancreatic tumor cells) stably expressing either wild-type human NIS polypeptides or human mutant NIS-93E polypeptides are implanted subcutaneously in the flanks of athymic mice. Once tumors grow to a diameter of 5-8 mm, ¹²³I/^{99m}Tc subtraction SPECT is performed using a dual-detector SPECT camera. Four hours after the intraperitoneal administration of 250 μCi ¹²³I sodium iodide and 250 μCi ^{99m}TcO₄, SPECT/CT data are acquired. The SPECT emission data are collected in dual-energy windows to separate the ^{99m}Tc and ¹²³I counts. The ^{99m}Tc window is centered at 140 keV, and the ¹²³I window is placed with a 4% offset above 159 keV to minimize the spillover of the ⁹⁹mTc photopeak into the much less intense ¹²³I photopeak. SPECT reconstruction is performed for each of the two acquisition windows. ¹²³I SPECT voxel intensities on thyroid tissue are normalized to the corresponding ^{99m}Tc SPECT data, and in this way a normalization factor is determined Each voxel in the ¹²³I SPECT dataset is then multiplied by this normalization factor, and the normalized ¹²³I SPECT data is subtracted from the ⁹⁹mTc SPECT data to create a subtraction SPECT dataset. Co-registration of the attenuation-corrected subtraction SPECT images with the CT images is performed using fusion software. A positive finding is defined as focal residual activity on the attenuation-corrected subtraction SPECT image that corresponds to non-thyroidal soft-tissue on CT images.

Subtraction images are presented to highlight the tumors expressing NIS-93E polypeptides, but not the tumors expressing wild-type NIS polypeptides. This is because the tumors expressing NIS-93E polypeptides concentrate only iodide and not pertechnetate such that image subtraction leaves a strong iodide uptake signal.

### Example 3

### Ingesting Oral CT Contrast Agents Removes Background Signals from the Stomach

Ingesting oral CT contrast agents (e.g., barium sulfate) removed background signals from the stomach as a result of NIS polypeptide-mediated uptake of radioisotopes, thus enhancing the quality of NIS imaging. In the following example, ingestion of barium sulfate was used to enable and enhance discrimination of NIS expression in the liver. The stomach endogenously expresses wild-type NIS polypeptides and uptakes radioisotopes such as radioiodine or pertechnetate, making the stomach highly visible in planar γ-camera, SPECT, or PET images. The strong stomach signal makes it difficult to define, detect, or pinpoint other NIS-positive cells in the surrounding area (e.g., the liver, perigastric area, pancreas, spleen, kidneys, or pleural space). However, a mouse fed barium sulfate by gavage and then imaged subsequently exhibited reduced isotope signals from the stomach and revealed positive isotope uptake by NIS polypeptide-expressing cells in the liver in a more apparent and defined manner (FIGS. 2A and 2B). These imaging results demonstrate that oral CT contrast agents can be used to achieve a substantial reduction of the stomach signal, giving confidence that the positive signals in the surrounding liver tissue are due to NIS polypeptide expression in the NIS transduced liver.

### Example 4

### Obtaining Mutated NIS Reporter Genes

Generating a Lentiviral NIS Expression Library of at Least 10⁵ NIS Variants with an Average of One to Two Amino Acid Mutations Per Variant

Library members are produced to be bicistronic, encoding NIS and YFP-H148Q/1152L, an iodide-sensitive reporter whose fluorescence is readily quenched by intracellular iodide, but not perchlorate. Library generation and characterization entails random PCR mutagenesis of the NIS nucleic acid, cloning the obtained nucleic acids into lentiviral vector plasmids, lentivirus library rescue, functional testing of library members, and determination of library diversity.

Error prone PCR (EP-PCR) is used to introduce random mutations into human NIS encoded in a plasmid. The 5′ and 3′ boundaries of the mutated NIS gene are defined by PCR primers encoding restriction sites required for cloning into a lentiviral transfer vector. A sample of the PCR product is cloned using the TOPO T/A cloning kit, and miniprep DNA is sequenced to determine the mutation frequency. If not satisfactory, the EP PRC is optimized. If satisfactory, the remainder of the PCR product is digested and cloned into a bistronic lentiviral vector encoding two different promoters: SFFV and CMV, driving the expression of HA-hNIS variant and YFP-H148Q/I152L, respectively (FIG. 3). The ligation mixture is used to transform E. coli, and lawns of ampicillin resistant bacteria are pooled and processed by Maxiprep (Qiagen) to purify the lentiviral library DNA.

### Lentivirus Library Rescue and Titration

Recombinant retrovirus particles are prepared by FuGENE transfection of HEK 293T cells with pantropic VSV-G encoding plasmid (pMDG), gag-pol-rev-tat encoding plasmid (R8.91) and transfer library plasmid DNA. Infectious particles are recovered, filtered, and concentrated using Centricon Ultracel PL-30 tubes (Millipore). The titer (TU/mL) of the lentiviral library is determined as a function of the percentage of infected cells expressing YFP-H148Q/I152L via flow cytometry.

Determining the Percentage of hNIS Variants that are Displayed on the Cell Surface and Transport Iodide into the Cell

To determine the percentage of hNIS variants that are displayed on the cell surface, cells are infected at a moi of 1. 72 hours post infection, the cells are subjected to two color flow cytometry to determine the percentage of cells that express YFP-H148Q/I152L and are tagged with anti-HA antibody conjugated to Alexa Fluor 647. To determine the percentage hNIS variants that can transport iodide into the cell, infected cells are treated with 200 μM NaI. The quenching of YFP-H148Q/I152L fluorescence (output for intracellular iodide uptake) is analyzed by flow cytometry.

### Selecting a Variant Whose Ability to Transport Iodide or Pertechnetate is Relatively Resistant to Perchlorate Inhibition

Cells fluorescing brightly after library infection are exposed to iodide in the presence of perchlorate. Flow sorting is used to select those cells whose fluorescence is quenched. Mutant NIS genes coding for perchlorate resistant NIS polypeptides are recovered from selected cells by PCR amplification and are further characterized.

Five million cells are infected at a moi of 1 with the lentiviral library transducing HA-hNIS variants. On the third day post infection, the cells are expanded to increase the number of cells expressing each unique NIS variant to limit the potential of it being lost during the selection procedure. On day 5, infected cells that fluoresce brightly are selected for by fluorescence-activated cell sorting (FACS) and collected in HBSS/HEPES buffer Immediately thereafter, these cells are incubated for 5 minutes with KClO₄ at a concentration that inhibits iodide transport by non-mutated NIS. Then, NaI is added to the cells to a finale concentration of 200 mM, and the cells are incubated for 10 minutes at 37° C. Cells whose fluorescence is quenched in the presence of KClO₄ are selected for by flow sorting. Selected cells are expanded, and stocks are frozen for further analysis and isolation of mutant NIS genes coding for perchlorate resistant NIS proteins. FIG. 3 (AIM2 section) demonstrates the inhibition of iodide mediated quenching of YFP-H148Q/I152L by 5 mM KClO₄. This concentration is optimized to inhibit quenching mediated by 200 mM NaI prior to the screen.

### Analysis of Isolated Cells Whose YFP-H148Q/I152L Fluorescence is Quenched in the Presence of Perchlorate

Results from above are confirmed using two additional assays for iodide uptake. First, a plate YFP-H148Q/I152L fluorescence iodide uptake assay is performed. Briefly, a fluorescent plate reader is used to measure the quenching of YFP-H148Q/I152L fluorescence following pretreatment of the cells with KClO₄ prior to the addition of cold iodide. Second, a radiotracer ¹²⁵I uptake assay is used. Briefly, cells are incubated with ¹²⁵I in the presence and absence of KClO₄ for 1 hour, washed twice with cold buffer, and lysed with NaOH. ¹²⁵I in cellular lysates is quantified using a gamma counter. Both these assays are performed on the cells while they are adhered to a culture plate, as opposed to the cells being in suspension during the FACS experiment, making the environment potentially less stressful.

Recovery of Mutant NIS Sequences Coding for Perchlorate Resistant NIS Polypeptides and their Characterization

Perchlorate resistant NIS variant genes are PCR amplified from cellular DNA using primers specific to the invariant 5′ and 3′ regions of the HA-NIS sequence. The PCR product is cloned using the TOPO T/A cloning kit, and miniprep DNA is sequenced to determine the mutation(s) encoded in the selected NIS variants. The recovered sequences coding for NIS mutants are then cloned back into the bisistronic lentiviral vector, and VSVG pseudotyped lentiviral vectors are produced in HEK 293T cells. Mel624 cells are infected at a moi of 10, and iodide uptake in the presence of KClO₄ is monitored using the two different assays described above.

### Example 5

### Subtractive Imaging

Subtractive imaging was performed to image a NIS expressing tumor, which concentrates both isotopes (¹²⁵I/^{99m}Tc). The stomach and thyroid signals were subtract from the SPECT dataset (FIG. 5). In this experiment, nude female mice (5-week of age) were injected with 3×10⁶ BXPC-3-hNIS cells. When tumors reached a diameter of 1 cm, mice were injected I.P. with ¹²⁵I sodium iodide (250 μCi) and/or ^{99m}Tc-pertechneate (250 μCi). Four hours post isotope administration, SPECT/CT data were acquired with U-SPECT-II (MiLabs). The SPECT emission data were collected in dual-energy windows to separate the gamma emision energy counts from ^{99m}Tc (140 keV) and ¹²⁵I (35 keV). FIG. 6 shows the uptake of ^{99m}Tc (green) and/or ¹²⁵I (red) by endogenous NIS expressing organs (thyroid and stomach) and heterologous NIS expressing tumor cells. Dual isotope imaging four hours after isotope administration (FIG. 6C) clearly revealed that thyroid preferentially accumulates ¹²⁵I (red), the stomach preferentially accumulates ^{99m}Tc (green), and the tumor cells accumulate both isotopes to similar concentrations (¹²⁵I (red)+^{99m}Tc (green)=yellow). Because different NIS expressing organs and tumor cells differed in the concentration of ¹²⁵I and ^{99m}Tc they accumulate following uptake, a subtraction technique was implemented and used to subtract the signal from the stomach (FIG. 5B) or thyroid (FIG. 5C) from the dataset (FIG. 5A). Radioiodide thyroid signal subtraction was performed by normalizing the ¹²⁵I SPECT data to thyroid tissue on the ⁹⁹mTc SPECT data using the image arithmetic tools available in PMOD image fusion software. This was performed in three steps by (1) calculating the normalization factor (i.e., the inverse ratio between the maximum voxel value corresponding to the thyroid tissue on the ¹²⁵I SPECT and the maximum voxel value corresponding to the thyroid tissue on the ^{99m}Tc SPECT data set), (2) multiplying each voxel in the ¹²⁵I SPECT dataset by this normalization factor, and (3) subtracting the normalized ¹²⁵I SPECT dataset from ^{99m}Tc SPECT data set to generate a thyroid subtraction SPECT dataset (FIG. 5C). Similarly, ⁹⁹mTc stomach signal subtraction was performed by (1) calculating the normalization factor which in this case is the inverse ratio of the maximum voxel value corresponding to the stomach tissue on the ⁹⁹mTc SPECT to the maximum voxel value corresponding to the stomach tissue in the ¹²⁵I SPECT data set, (2) multiplying each voxel in the ^{99m}Tc SPECT dataset by this normalization factor, and (3) subtracting the normalized ^{99m}Tc SPECT dataset from ¹²⁵I SPECT data set to generate a stomach subtraction SPECT dataset (FIG. 5B).

Tumors expressing NIS-93E polypeptides, but not the tumors expressing wild-type NIS polypeptides are additionally highlighted. This is because the tumors expressing NIS-93E polypeptides concentrate only iodide and not pertechnetate such that image ^{99m}Tc signal subtraction leaves a strong iodide uptake signal.

To increase the resolution of NIS expressing tumor cells following stomach subtraction SPECT, a NIS mutant that selectively transports radioiodine, but not ^{99m}Tc, is engineered as described herein and is used as described herein.

For example, stomach signal subtraction is performed using a NIS-G93E mutant or another NIS mutant provided herein by (1) calculating the normalization factor which can be the inverse ratio of the maximum voxel value corresponding to the stomach tissue on the ^{99m}Tc SPECT to the maximum voxel value corresponding to the stomach tissue in the ¹²⁵I SPECT data set, (2) multiplying each voxel in the ^{99m}Tc SPECT dataset by this normalization factor, and (3) subtracting the normalized ^{99m}Tc SPECT dataset from ¹²⁵I SPECT data set to generate a stomach subtraction SPECT dataset.

A NIS mutant, NIS-G93E, was determined to transport selectively Iodine-125 (FIG. 1B), but not Technetium-99m (FIG. 1D). In addition, the G93E mutation was found to decrease the concentration of Iodine-125 uptake in tumor cells as compared to non-mutated NIS (NIS WT) (FIGS. 1A and 1B).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of imaging an animal comprising cells expressing a NIS transgene, wherein said method comprises:
(a) administering two or more gamma-emitting NIS radiotracers to said animal,
(b) collecting imaging data during a single imaging session using at least two energy windows that distinguish the gamma emissions of said two or more radiotracers to obtain at least two gamma emission datasets,
(c) subtracting one of said at least two gamma emission datasets from another of said at least two gamma emission datasets to obtain a resulting dataset capable of being used to generate subtraction images for visual or other analysis.

2. The method of claim 1, wherein one of said at least two gamma emission datasets is pseudo-colored in one color, another of said at least two gamma emission datasets is pseudo-colored in another color, and wherein said at least gamma emission datasets are digitally merged to generate one or more pseudo-color images for visual analysis.

3. The method of claim 1, wherein said NIS transgene encodes a mutated NIS polypeptide with diminished capacity to concentrate one or more NIS radiotracers.

4. The method of claim 1, wherein said NIS transgene encodes a mutated NIS polypeptide with diminished capacity to concentrate pertechnetate.

5. The method of claim 4, wherein said mutant NIS polypeptide is NIS-93E.

6. A method of imaging an animal comprising cells expressing a NIS transgene, wherein said method comprises:
(a) administering a gamma-emitting or positron-emitting NIS radiotracer to said animal,
(b) administering non-radioactive perchlorate anions to said animal in a dose sufficient to substantially inhibit the uptake of said radiotracer by endogenous NIS-expressing cells, and
(c) collecting imaging data from said animal.

7. The method of claim 6, wherein said NIS transgene encodes a mutated NIS polypeptide whose ability to concentrate NIS radiotracers has reduced susceptibility to perchlorate inhibition in comparison to a non-mutated NIS polypeptide.

8. A method for imaging a mammal to reduce background from cells endogenously expressing a wild type NIS polypeptide within the stomach of said mammal, wherein said method comprises obtaining an image of radioisotope signals from a radioisotope present within said mammal, wherein said image is obtained within four hours of said mammal ingesting a contrast agent, wherein cells outside the stomach of said mammal expressing a NIS polypeptide uptake said radioisotope.

9. The method of claim 8, wherein said radioisotope is pertechnetate or radioiodide.

10. The method of claim 8, wherein said contrast agent is barium sulphate.

11. A method for imaging a mammal to reduce background from radioisotope signals from cells endogenously expressing a wild-type NIS polypeptide, wherein said mammal comprises cells endogenously expressing said wild-type NIS polypeptide and cells expressing a mutant NIS polypeptide, wherein said method comprises:
(a) obtaining a first image of radioisotope signals from a first radioisotope present within a mammal, wherein cells endogenously expressing said wild-type NIS polypeptide within said mammal uptake said first radioisotope to a greater extent than cells expressing said mutant NIS polypeptide,
(b) obtaining a second image of radioisotope signals from a second radioisotope present within a mammal, wherein cells endogenously expressing said wild-type NIS polypeptide within said mammal and cells expressing said mutant NIS polypeptide within said mammal uptake said second radioisotope, and
(c) removing radioisotope signals of said first image from the radioisotope signals of said second image to obtain a final image.

12. The method of claim 11, wherein said mammal is a human.

13. The method of claim 11, wherein said wild-type NIS polypeptide is a human NIS polypeptide.

14. The method of claim 11, wherein said mutant NIS polypeptide is a NIS-93E polypeptide or a NIS-93Q polypeptide.

15. The method of claim 11, wherein said mutant NIS polypeptide is a NIS-93E polypeptide.

16. The method of claim 11, wherein said first radioisotope is pertechnetate.

17. The method of claim 11, wherein said second radioisotope is radioiodide.

18. The method of claim 11, wherein cells expressing said mutant NIS polypeptide within said mammal do not uptake said first radioisotope.

19. The method of claim 11, wherein cells endogenously expressing said wild-type NIS polypeptide and cells expressing said mutant NIS polypeptide uptake said second radioisotope with substantially different efficiencies.

20. The method of claim 11, wherein method comprises removing substantially all radioisotope signals of said first image from the radioisotope signals of said second image to obtain said final image.
